(19) United States
(12) Patent Application Publication
(10) Pub. No.: US 2022/0257626 A1
(43) Pub. Date: Aug. 18, 2022

(54) TREATMENT COMPRISING SGLT INHIBITORS

(71) Applicant: Novartis AG, Basel (CH)

(72) Inventors: Dominique BREES, Arlesheim (CH); Patricia LOPEZ, Basel (CH)

(21) Appl. No.: 17/628,857

(22) PCT Filed: Jul. 21, 2020
(86) PCT No.: PCT/IB2020/056838

§ 371 Date: Jan. 20, 2022

Related U.S. Application Data
(60) Provisional application No. 62/877,464, filed on Jul. 23, 2019.
(60) Provisional application No. 62/901,418, filed on Sep. 17, 2019.

(51) Int. Cl. A61K 31/7048; A61P 1/16

(52) U.S. Cl. CPC A61K 31/7048; A61P 1/16

(57) ABSTRACT

The invention relates to methods for treating, preventing, or ameliorating a liver disease, e.g. NASH, comprising administering to a subject in need thereof a therapeutically effective amount of a SGLT inhibitor, e.g. SGLT 1/2 inhibitor.

### FIELD OF THE INVENTION

The present invention relates to pharmaceutical uses of licogliflozin, its pharmaceutically acceptable salts, and prodrugs thereof, for the treatment of a liver disease or disorder, or an intestinal disease, specifically for the treatment of non-alcoholic steatohepatitis (“NASH”).

### BACKGROUND OF THE INVENTION

Nonalcoholic fatty liver disease (NAFLD) is the most common cause of chronic liver disease in the Western world. The main stages of NAFLD are 1—simple fatty liver (steatosis); 2—non-alcoholic steatohepatitis (NASH), a more serious form of NAFLD with fat accumulation accompanied by inflammation and cell injury; 3—fibrosis, where there is a persistent inflammation in the liver resulting in the generation of fibrous scar tissue around the liver cells and blood vessels; and 4-cirrhosis; this damage is permanent and can lead to liver failure and liver cancer (hepatocellular carcinoma).

Liver transplantation is the only treatment for advanced cirrhosis with liver failure. Estimates of the worldwide prevalence of NAFLD range from 6.3% to 33% with a median of 20% in the general population. The estimated prevalence of NASH is lower, ranging from 3 to 5% (Younossi et al., Hepatology, Vol. 64, No. 1, 2016). NASH is a worldwide problem with growing prevalence over the last few decades. Over the last decade NASH has risen from uncommon to the second indication for liver transplantation in the US. It is expected to be the leading cause of transplant by 2024. NASH is highly associated with the metabolic syndrome and Type 2 diabetes mellitus. Furthermore, cardiovascular mortality is an important cause of death in NASH patients.

Development of NASH, involves several mechanisms: accumulation of fat in the liver (steatosis), inflammation of the liver, hepatocyte ballooning, and fibrosis. The NAFLD Activity Score (NAS) was developed as a tool to measure changes in NAFLD during therapeutic trials. The score is calculated as the unweighted sum of the scores for steatosis (0-3), lobular inflammation (0-3), and ballooning (0-2).

Obesity has become a major global health problem that contributes causally to and exacerbates many serious co-morbidities including hypertension, dyslipidemia, type 2 diabetes (T2DM) and importantly non-alcoholic fatty liver disease (NAFLD). In support of the link between obesity and fatty liver linked hepatic injury, weight loss either through bariatric surgery, diet or exercise leads to improvement in histologic NASH. This suggests that targeting obesity in NASH patients is likely to limit or reverse liver disease progression. A novel mechanism to lower body weight is via inhibition of the sodium glucose co-transporters 1 and 2 (SGLTs) resulting in inhibition of the glucose absorption in the gut and reabsorption in the kidney.

Licogliflozin (also known as LIK066) is (2S,3R,4R,5S,6R)-2-(3-((2,3-dihydrobenzo[b][1,4]dioxin-6-yl)methyl)-4-ethylphenyl)-6-(hydroxymethyl)tetrahydro-2H-pyran-3,4,5-triol, has the following chemical structure:

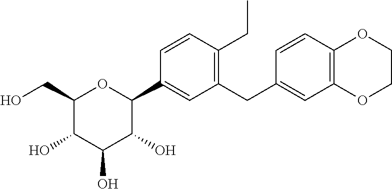 [SMILES: CCc1ccc([C@@H]2O[C@H](CO)[C@@H](O)[C@H](O)[C@H]2O)cc1Cc1ccc2c(c1)OCCO2]

Licogliflozin is a potent inhibitor of the sodium glucose co-transporters (SGLTs) 1 and 2 that decreases absorption of glucose in the gut and reabsorption in the kidney. Licogliflozin was found to be safe and tolerated, had a favorable pharmacokinetic profile, and resulted in up to 3% placebo-adjusted weight loss over just 2 weeks in both healthy subjects and patients with T2DM. Licogliflozin at 150 mg daily dose results in a significant weight loss in obese patients (˜6%) after 12 week treatment. Furthermore, twelve week treatment with licogliflozin at 150 mg once daily, in normoglycemic and dysglycemic subjects was generally safe and well tolerated with diarrhea observed as a dose-limiting toxicity.

Currently there is no approved therapy for NASH. Therefore, there is a need to provide treatments for fibrotic/cirrhotic diseases or disorders, e.g. liver diseases or disorders, e.g. NASH, which can address the different aspects of these complex conditions, while demonstrating an acceptable safety and/or tolerability profile.

### SUMMARY OF THE INVENTION

The invention relates to methods for treating, preventing, or ameliorating a liver disease, e.g. NASH, comprising administering to a subject in need thereof a therapeutically effective amount of a SGLT inhibitor, e.g. SGLT 1/2 inhibitor, wherein the administration of a SGLT inhibitor to said subject is occurring in the evening.

The invention provides new treatment regimens for pharmaceutical compositions containing a SGLT 1 and/or 2 inhibitor. The treatment regimens, according to the present, invention offer the benefit of a high therapeutic efficacy while having low incidence of side effects, such as diarrhea, which are, observed while using conventional treatment regimen. These treatment regimens further provide subjects with a convenient once daily dosing, thus supporting patient compliance.

### DETAILED DESCRIPTION OF THE INVENTION

Various (enumerated) embodiments of the present invention are described herein. It will be recognized that features specified in each embodiment may be combined with other specified features to provide further embodiments of the present disclosure.

### EMBODIMENTS (a)

1a: A SGLT inhibitor, e.g. SGLT 1/2 inhibitor, for use in the treatment of a liver disease, e.g NASH, wherein the SGLT inhibitor is administered once daily at a therapeutically effective dose, and wherein the SGLT inhibitor is administered in the evening.

2a: A SGLT inhibitor, e.g. SGLT 1/2 inhibitor, for use in the prevention of a liver disease, e.g NASH, wherein the SGLT inhibitor is administered once daily at a therapeutically effective dose, and wherein the SGLT inhibitor is administered in the evening.

3a. A SGLT inhibitor, e.g. SGLT 1/2 inhibitor, for use in the treatment, stabilization or lessening the severity or progression of a non-alcoholic fatty liver disease (NAFLD), e.g. NASH, in a subject in need thereof, wherein the SGLT inhibitor is administered once daily at a therapeutically effective dose, and wherein the SGLT inhibitor is administered in the evening.

4a. A SGLT inhibitor, e.g. SGLT 1/2 inhibitor, for use in the treatment, stabilization or lessening the severity or progression of an intestinal disease in a subject in need thereof, wherein the SGLT inhibitor is administered once daily at a therapeutically effective dose, and wherein SGLT inhibitor is administered in the evening.

5a. A SGLT inhibitor, e.g. SGLT 1/2 inhibitor, for use in the slowing, arresting, or reducing the development of a chronic liver disease or disorder, e.g. NAFLD, NASH, liver fibrosis or PBC, in a subject in need thereof, wherein the SGLT inhibitor is administered once daily at a therapeutically effective dose, and wherein the SGLT inhibitor is administered in the evening.

6a. The SGLT inhibitor, e.g. SGLT 1/2 inhibitor, according to any one of Embodiments 1a to 5a, wherein the SGLT inhibitor is selected from licogliflozin, dapagliflozin, canagliflozin, empagliflozin, ipragliflozin, ertugliflozin, mizagliflozin, and sotagliflozin.

7a. The SGLT inhibitor, e.g. SGLT 1/2 inhibitor, according to Embodiment 6a, wherein the SGLT inhibitor is licogliflozin.

8a. The licoglifozin according to Embodiment 7a, wherein licogliflozin is administered once daily at an amount of about 30 mg or of about 150 mg.

9a. Licoglifozin, e.g. in free form, or a salt thereof, for use in the treatment or prevention of a liver disease, e.g NASH, wherein licoglifozin is administered once daily, at a therapeutically effective dose, and wherein licoglifozin is administered in the evening.

10a. Licoglifozin, e.g. in free form, or a salt thereof, for use in the treatment or prevention of non-alcoholic fatty liver disease (NAFLD), non-alcoholic steatohepatitis (NASH), liver fibrosis or PBC, wherein licoglifozin is administered once daily, at a therapeutically effective dose, and wherein licoglifozin is administered in the evening.

11a. Licoglifozin, e.g. in free form, or a salt thereof, or an amino acid conjugate thereof, for use in the treatment or prevention of non-alcoholic fatty liver disease (NAFLD), or of non-alcoholic steatohepatitis (NASH), wherein licoglifozin is to be administered once daily, at a dose of about 20 mg to about 200 mg, or of about 30 mg to about 150 mg, and wherein licoglifozin is administered in the evening.

12a. Licoglifozin for use according to any of Embodiments 7a to 11a, wherein licoglifozin is to be administered at a daily dose of about 30 mg.

13a. The SGLT inhibitor, e.g. SGLT 1/2 inhibitor, for use according to any one of Embodiments 1a to 12a, wherein said evening administration ameliorates the efficacy associated with the administration of the SGLT inhibitor, e.g. SGLT 1/2 inhibitor.

14a. The SGLT inhibitor, e.g. SGLT 1/2 inhibitor, for use according to any one of Embodiments 1a to 13a, wherein said evening administration reduces the risk of side effects, e.g. diarrhea, associated with the administration of the SGLT inhibitor, e.g. SGLT 1/2 inhibitor.

15a. The SGLT inhibitor, e.g. SGLT 1/2 inhibitor, for use according to any one of Embodiments 1a to 14a, wherein said administration comprises resolution of steatohepatitis.

16a. The SGLT inhibitor, e.g. SGLT 1/2 inhibitor, for use according to any one of Embodiments 1a to 14a, wherein said administration comprises improvement in liver fibrosis.

17a. The SGLT inhibitor, e.g. SGLT 1/2 inhibitor, for use according to any one of Embodiments 1a to 14a, wherein said administration comprises resolution of steatohepatitis and improvement in liver fibrosis.

### EMBODIMENTS (b)

1b. A method for the treatment of a liver disease, in a subject in need thereof, comprising administering once daily to said subject a therapeutically effective amount of a SGLT inhibitor, e.g. SGLT 1/2 inhibitor, wherein the SGLT inhibitor, e.g. SGLT 1/2 inhibitor, is administered in the evening.

2b. A method for the prevention of a liver disease in a subject in need thereof, comprising administering once daily to said subject a therapeutically effective amount of a SGLT inhibitor, e.g. SGLT 1/2 inhibitor, wherein the SGLT inhibitor, e.g. SGLT 1/2 inhibitor, is administered in the evening.

3b. A method for the treatment, stabilization or lessening the severity or progression of a non-alcoholic fatty liver disease (NAFLD) in a subject in need thereof, comprising administering once daily to said subject a therapeutically effective amount of a SGLT inhibitor, e.g. SGLT 1/2 inhibitor, wherein the SGLT inhibitor, e.g. SGLT 1/2 inhibitor, is administered in the evening.

4b. A method for the treatment, stabilization or lessening the severity or progression of an intestinal disease in a subject in need thereof, comprising administering once daily to said subject a therapeutically effective amount of a SGLT inhibitor, e.g. SGLT 1/2 inhibitor, wherein the SGLT inhibitor, e.g. SGLT 1/2 inhibitor, is administered in the evening.

5b. A method for the treatment, stabilization or lessening the severity or progression of a non-alcoholic steatohepatitis (NASH) in a subject in need thereof comprising administering once daily to said subject a therapeutically effective amount of a SGLT inhibitor, e.g. SGLT 1/2 inhibitor, wherein the SGLT inhibitor, e.g. SGLT 1/2 inhibitor, is administered in the evening.

6b. A method for slowing, arresting, or reducing the development of a chronic liver disease or disorder, e.g. NAFLD, NASH, liver fibrosis or PBC, in a subject in need thereof, comprising administering once daily to said subject a therapeutically effective amount of a SGLT inhibitor, e.g. SGLT 1/2 inhibitor, wherein the SGLT inhibitor, e.g. SGLT 1/2 inhibitor, is administered in the evening.

7b. A method for reducing cirrhosis or fibrosis in a subject having a disease that is non-alcoholic fatty liver disease (NAFLD) or non-alcoholic steatohepatitis (NASH), comprising administering once daily to said subject a therapeutically effective amount of a SGLT inhibitor, e.g. SGLT 1/2 inhibitor, wherein the SGLT inhibitor, e.g. SGLT 1/2 inhibitor, is administered in the evening.

8b. The method according to any one of Embodiments 1b to 7b, wherein said method further comprises lack of worsening of the subject's NAFLD as defined by Activity (NAS) score, lack of worsening of the subject's Steatosis, Activity and Fibrosis (SAF) Activity score, reduction of liver fat in said subject, improvement in subject's Steatosis, improvement in subject's ballooning, NAFLD resolution, NAFLD resolution without worsening of fibrosis, reduction of fibrosis without NAFLD worsening, reduction of ALT levels in said subject, reduction of AST levels in said subject, reduction of HbA1c levels in said subject, lack of subject's progression to Cirrhosis, inhibiting progression of Non-Alcoholic Fatty Liver Disease (NAFLD) and/or Non-Alcoholic Steatohepatitis (NASH), or any combination thereof.

9b. The method according to any one of Embodiments 1b to 8b, wherein the SGLT inhibitor, e.g. SGLT 1/2 inhibitor, is selected from licogliflozin, dapagliflozin, canagliflozin, empagliflozin, ipragliflozin, ertugliflozin, mizagliflozin, and sotagliflozin.

10b. The method according to Embodiment 9b, wherein the SGLT inhibitor, e.g. SGLT 1/2 inhibitor, is licogliflozin.

11b. The method according to Embodiment 10b, wherein licogliflozin is administered at a daily dose from about 30 mg to about 150 mg, preferably at about 30 mg.

12b. The method according to any one of Embodiments 1 b to 11 b, wherein said evening administration ameliorates the efficacy associated with administration of the SGLT inhibitor, e.g.

SGLT 1/2 inhibitor.

13b. The method according to any one of Embodiments 1b to 12b, wherein said evening administration reduces the risk of side effects, e.g. diarrhea, associated with administration of the SGLT inhibitor, e.g. SGLT 1/2 inhibitor.

14b. The method according to any one of Embodiments 1b to 13b, wherein said administration comprises resolution of steatohepatitis, e.g. NASH.

15b. The method according to any one of Embodiments 1b to 13b, wherein said administration comprises improvement in liver fibrosis.

16b. The method according to any one of Embodiments 1b to 13b, wherein said administration comprises resolution of steatohepatitis, e.g. NASH, and improvement in liver fibrosis.

There is provided the use of licogliflozin (or a pharmaceutically acceptable salt, solvate, prodrug and/or ester thereof), for the manufacture of a medicament for the prevention or treatment of a liver disease or disorder, e.g. a liver disease or disorder selected from the group consisting of NAFLD, NASH, hepatosteatosis, liver fibrosis, cirrhosis, PBC.

There is also provided pharmaceutical composition containing licogliflozin in an amount of about 20 to about 200 mg. Preferably, in an amount of about 30 mg to about 150 mg, e.g. in an amount of about 30 mg.

There is also provided a SGLT inhibitor, e.g. SGLT 1/2 inhibitor according to any one of the above listed Embodiments, for treating or preventing non-alcoholic steatohepatitis (NASH), and wherein NASH is mild to moderate with fibrosis level F2-F3.

A SGLT inhibitor, e.g. SGLT 1/2 inhibitor, according to any one of above listed Embodiments, wherein NASH is confirmed based on liver biopsy (also called biopsy-proven NASH) and NASH is mild to moderate with fibrosis level F2-F3.

A SGLT inhibitor, e.g. SGLT 1/2 inhibitor, according to any one of the above listed Embodiments, wherein presence of NASH has been demonstrated by:
i) Histologic evidence of NASH based on liver biopsy obtained 2 years or less before treatment with a FXR agonist according to any one of the above Embodiments, with a diagnosis consistent with NASH, fibrosis level F1, F2, F3 or F4, no diagnosis of alternative chronic liver diseases, or
ii) Phenotypic diagnosis of NASH, or
iii) Noninvasive, disease-specific biomarkers.

According to the invention, licogliflozin (as hereinabove defined) is administered at a dose of about 20 mg to about 200 mg, e.g. about 30 mg, e.g. about 40 mg, e.g. about 50 mg, e.g. about 60 mg, e.g. about 70 mg, e.g. about 80 mg, e.g. about 90 mg, e.g. about 100 mg, e.g. about 120 mg, e.g. about 150 mg. Such doses may be for daily oral administration of licogliflozin.

According to the invention, licogliflozin (as hereinabove defined) is administered at a dose of about 30 mg, once daily, e.g. in the evening.

In some aspects, the pharmaceutical combinations, as defined herein, are provided for the treatment of a liver disease or disorder, e.g. a chronic liver disease or disorder, e.g. a disease or disorder selected from the group consisting of cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g. NAFLD, NASH, hepatic fibrosis, hepatosteatis or PBC.

### Definitions

For purposes of interpreting this specification, the following definitions will apply and whenever appropriate, terms used in the singular will also include the plural and vice versa.

As used herein, the term “about” in relation to a numerical value x means +/−10%, unless the context dictates otherwise.

As used herein, a “SGLT inhibitor” refers to any agent that is capable of inhibiting SGLT, e.g. individual SGLT1 and SGLT2 inhibitors, as well as dual SGLT1/2 inhibitors. The SGLT inhibitor as used herein refers, for example, to licogliflozin, dapagliflozin, canagliflozin, empagliflozin, ipragliflozin, ertugliflozin, mizagliflozin, sotagliflozin.

Sotagliflozin is (2S,3R,4R,5S,6R)-2-[4-chloro-3-[(4-ethoxyphenyl)methyl]phenyl]-6-methylsulfanyloxane-3,4,5-triol, also known as LX4211.

As used herein, the terms “salt” or “salts” refer to an acid addition or base addition salt of a compound of the invention. “Salts” include in particular “pharmaceutical acceptable salts”, and both can be used interchangeably herein.

As used herein, the term “pharmaceutically acceptable” means a nontoxic material that does not substantially interfere with the effectiveness of the biological activity of the active ingredient(s).

As used herein the term “prodrug” refers to a compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the terms “subject” or “subjects” refer to a mammalian organism, preferably a human being, who is diseased with the condition (i.e. disease or disorder) of interest and who would benefit from the treatment, e.g. a patient.

As used herein, a subject is “in need of” a treatment if such subject would benefit biologically, medically or in quality of life from such treatment.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter), or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition, such as e.g. liver cirrhosis; or to preventing or delaying a need for liver transplantation.

As used herein, the term nonalcoholic fatty liver disease (NAFLD) may refer to nonalcoholic fatty liver (NAFL), non-cirrhotic NASH, and NASH with cirrhosis.

For example, “treating” NASH may refer to ameliorating, alleviating or modulating at least one of the symptoms or pathological features associated with NASH; e.g. hepatosteatosis, hepatocellular ballooning, hepatic inflammation and fibrosis; e.g. may refer to slowing progression, reducing or stopping at least one of the symptoms or pathological features associated with NASH, e.g. hepatosteatosis, hepatocellular ballooning, hepatic inflammation and fibrosis. It may also refer to preventing or delaying liver cirrhosis or a need for liver transplantation, e.g. slow the progress of, halt, or reverse disease progression and improve clinical outcomes (i.e., prevent progression to cirrhosis and 283 cirrhosis complications, reduce the need for liver transplantation, and improve survival).

Also “treating” NASH may refer to slow the progress of, halt, or reverse disease progression and improve clinical outcomes i.e., prevent progression to cirrhosis and Resolution of steatohepatitis and no worsening of liver fibrosis on NASH clinical research network (CRN) histological score.

The treatment of NASH includes:
“Resolution of steatohepatitis” is defined as absence of fatty liver disease or isolated or simple steatosis without steatohepatitis and a NAS score of 0-1 for inflammation, 0 for ballooning, and any value for steatosis; cirrhosis complications, reduction in the need for liver transplantation, and improved survival;
Or Improvement in liver fibrosis greater than or equal to one stage (NASH CRN histological score) and no worsening of steatohepatitis (e.g. defined as no increase in NASH for ballooning, inflammation, or steatosis);
Or Both resolution of steatohepatitis and improvement in fibrosis (as defined above).

“Treating” or “treatment” of NAFLD or NASH in a human includes one or more of:
a) Reducing the risk of developing NAFLD or NASH, i.e., causing clinical symptoms of NAFLD or NASH not to develop in a subject who may be predisposed to NAFLD, or NASH;
b) Inhibiting NAFLD or NASH, i.e., arresting or reducing the development of NALFD, or NASH, or its clinical symptoms; and
c) Relieving NAFLD or NASH, i.e., causing regression, reversal, or amelioration of the NAFLD, or NASH, or reducing number, frequency, duration or severity of its clinical symptoms.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

As used herein, the term “therapeutically effective amount” refers to an amount of the compound, which is sufficient to achieve the stated effect. Accordingly, a therapeutically effective amount used for the treatment or prevention of a liver disease or disorder, as hereinabove defined, is an amount sufficient for the treatment or prevention of such a disease or disorder.

By “therapeutic regimen” is meant the pattern of treatment of an illness, e.g., the pattern of dosing used during the treatment of the disease or disorder.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term “NASH” may encompass steatosis, hepatocellular ballooning and lobular inflammation.

As used herein, the term “qd” means a once daily administration.

The term “dose” refers to a specified amount of a drug administered at one time. As used herein, the dose is the amount of the drug that elicits a therapeutic effect. The dose would, for example, be declared on a product package or in a product information leaflet.

### Modes of Administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

### Timing of the Administration

The SGLT inhibitor, e.g. SGLT 1/2 inhibitor, as defined herein in the above listed embodiments, is administered in the evening.

In one embodiment, the term “administration in the evening” is generally defined as administration any time from about 6 pm to about 12 pm, e.g. from about 8 pm to about 11 pm, preferably around 9 pm. Administration in the evening may be before the evening meal, with the evening meal or after the evening meal. Preferably, administration in the evening is with the evening meal.

In one embodiment, the term “administration in the evening” refers to administration shortly before or at bedtime. In one embodiment, the term “administration in the evening” refers to administration shortly before bedtime. In one embodiment, the term “administration in the evening” refers to administration at bedtime. Unless otherwise specified herein, the term “bedtime” has the normal meaning of a time when a person retires for the primary sleep period during a twenty-four hours period of time. The administration shortly before bedtime means that the SGLT inhibitor, e.g. SGLT 1/2 inhibitor as herein defined, is administered within about 30 minutes to about 2 hours prior to a person's normal rest or sleep (typically 4 to 10-hours) period.

### Diseases

As hereinabove defined, the fibrotic or cirrhotic disease or disorder can be a liver disease or disorder, e.g. as defined herein, or renal fibrosis.

As hereinabove defined, the liver diseases or disorders can be cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis. The liver diseases or disorders can also refer to liver transplantation.

As hereinabove defined, the intestinal disease can be idiopathic inflammatory bowel disease, e.g. Crohn's disease or ulcerative colitis.

In one embodiment of the invention, the pharmaceutical combinations (as herein defined) are for the treatment or prevention of a fibrotic disease or disorder, e.g. a liver disease or disorder, e.g. a chronic liver disease, e.g. a liver disease or disorder selected from the group consisting of PBC, NAFLD, NASH, drug-induced bile duct injury, gallstones, liver cirrhosis, alcohol-induced cirrhosis, cystic fibrosis-associated liver disease (CFLD), bile duct obstruction, cholelithiasis, liver fibrosis. In one embodiment of the invention, the pharmaceutical combination (as herein defined) is for the treatment or prevention of fibrosis, e.g. renal fibrosis or liver fibrosis.

According to one embodiment of the invention, the liver diseases or disorders refer to NAFLD, e.g. any stages of NAFLD, e.g. any of steatosis, NASH, fibrosis and cirrhosis.

In one embodiment of the invention, there is provided a SGLT inhibitor, e.g. SGLT 1/2 inhibitor of the invention, as herein defined in above listed embodiments, for the improvement of liver fibrosis without worsening of steatohepatitis.

In another embodiment of the invention, there is provided a SGLT inhibitor, e.g. SGLT 1/2 inhibitor of the invention, as herein defined in above listed embodiments, for obtaining a complete resolution of steatohepatitis without worsening, e.g. improving, of liver fibrosis.

In another embodiment of the invention, there is provided a SGLT inhibitor, e.g. SGLT 1/2 inhibitor of the invention, as herein defined in above listed embodiments, for preventing or treating steatohepatitis and liver fibrosis.

In yet another embodiment of the invention, there is provided a SGLT inhibitor, e.g. SGLT 1/2 inhibitor of the invention, as herein defined in above listed embodiments, for reducing at least one of the features of the NAS score, i.e. one of hepatosteatosis, hepatic inflammation and hepatocellular ballooning; e.g. at least two features of the NAS score, e.g. hepatosteatosis and hepatic inflammation, or hepatosteatosis and hepatocellular ballooning, or hepatocellular ballooning and hepatic inflammation.

In a further embodiment of the invention, there is provided a SGLT inhibitor, e.g. SGLT 1/2 inhibitor as herein defined in above listed embodiments, for reducing at least one or two features of the NAS score and liver fibrosis, e.g. for reducing hepatic inflammation and liver fibrosis, or hepatosteatosis and liver fibrosis or hepatocellular ballooning and liver fibrosis.

In yet a further embodiment of the invention there is provided a SGLT inhibitor, e.g. SGLT 1/2 inhibitor as herein defined, for treating or preventing, stage 3 fibrosis to stage 1 fibrosis, e.g. stage 3 and/or stage 2 and/or stage 1 fibrosis.

### Subjects

According to the invention, the subjects receiving the pharmaceutical combination of the invention can be affected or at risk of a fibrotic disease or disorder, e.g. a liver disease or disorder, as hereinabove defined.

In some embodiments of the invention, the subject is obese or overweight.

In other embodiments of the invention, the subject may be a diabetic subject, e.g. may have type 2 diabetes. The subject may have high blood pressure and/or high blood cholesterol level.

### Dosing Regimens

Depending on the compound used, the targeted disease or disorder and the stage of such disease or disorder, the dosing regimen, i.e. administered doses and/or frequency may vary. The dosing frequency will depend on; inter alia, the phase of the treatment regimen. The frequency of dosing of licogliflozin, may be once per day.

In one embodiment of the invention, the administration is carried out for at least one week, at least one month, at least six weeks, at least three months, at least six months, at least one year. For example, the invention is administered lifelong to the patient. The frequency of administration and/or the doses of licogliflozin may vary during the whole period of administration.

According to the invention, licogliflozin (as hereinabove defined) is administered at a dose of e.g. about 20 mg, e.g. about 30 mg, e.g. about 50 mg, e.g. about 60 mg, e.g. about 80 mg, e.g. about 90 mg, e.g. about 100 mg, e.g. about 120 mg, e.g. about 150 mg. Such doses may be for oral administration licogliflozin. Such doses may be for daily oral administration of licogliflozin.

In some aspects, licogliflozin is administered at a dose of about 30 mg. Such dose may be for daily oral administration licogliflozin.

### EXAMPLES

### Example 1: Clinical Study for Efficacy, Safety, and Tolerability in Subjects with NASH and Fibrosis (Stage 2 or 3) as Per NASH CRN Histological Score

Primary objective: To demonstrate the efficacy of licogliflozin as assessed by histologic improvement after 48 weeks of treatment in subjects with NASH and stage 2 or 3 fibrosis.

Secondary objectives:
Improvement in fibrosis by at least one stage with no worsening of NASH after 48 weeks of treatment
Resolution of NASH with no worsening of fibrosis after 48 weeks of treatment
Improvement in fibrosis by at least one stage
Improvement in fibrosis by at least two stages with no worsening of NASH after 48 weeks of treatment
Reduction in body weight from baseline after 48 weeks of treatment
Change in liver fat content after 48 weeks of treatment
To determine the relationship of investigational treatment and markers of hepatic inflammation in NASH (ALT and AST)
To determine the relationship of investigational treatment and GGT, a marker of cholestasis

The study consists of 1) a screening period, 2) a treatment period starting from randomization on Day 1 and running to Week 48, and 3) a follow up period of 4 weeks after the last dose of study treatment. The screening period starts from the time of the signing of informed consent and continues for up to 8 weeks when all inclusion/exclusion criteria have been evaluated and all baseline assessments have been performed. The study duration from first dose of study medication is 52 weeks. The total duration of participation may be up to 60 weeks.

Subjects eligible for inclusion in this study must meet all of the following criteria:
Written informed consent must be obtained before any assessment is performed.
Male and female subjects 18 years or older (at the time of the screening visit)
Presence of NASH as demonstrated by the following during the screening period: NASH with fibrosis stage 2 or 3 confirmed by central reader's evaluation using NAFLD Activity Score (NAS) and NASH CRN criteria, of liver biopsy obtained no more than 6 months before randomization.
Able to communicate well with the investigator, to understand and comply with the requirements of the study

The planned duration of treatment is 48 weeks. Subjects may be discontinued from treatment earlier due to unacceptable tolerability, disease progression and/or at the discretion of the investigator or the subject.

In a Phase 2 dose range finding study in obese patients, licogliflozin was associated with a dose-dependent increase in incidence of diarrhea (18.4%, 15.8%, 55.3%, 68.8%, following 2.5, 10, 50, and 150 mg QD for 24 weeks vs. 19.2% on placebo; CLIK066B2201). A dose of 30 mg QD is expected to achieve approximately 70% of maximum observed efficacy (using weight loss as a downstream marker for efficacy; CLIK066B2201). Licogliflozin is currently being tested as 30 mg and 150 mg QD monotherapy in NASH (CLIK066X2204). An interim analysis on the 150 mg QD treatment group showed promising reduction of ALT and liver fat, among other efficacy endpoints, but significant incidence of gastrointestinal events (mainly diarrhea). To minimize the risk of GI adverse effects of the SGLT1 inhibition in the gut such as diarrhea, licogliflozin will administered in the evening.

The doses for this study were selected based on the expectation of achieving increased efficacy with the combination therapy, compared to individual monotherapies, while maintaining tolerability and safety of the patients.

Subjects (n=70) are assigned at baseline visit to licogliflozin therapy Arm: licogliflozin 30 mg, once daily. Subjects should take the medication in the evening following a meal and at about the same time each day, except at baseline and week 4 where the dose will be taken in the morning at the clinic instead of evening dose.

The efficacy assessments should be completed in the following recommended order:
MRI.
Liver function test: ALT, AST, GGT, total alkaline phosphatase (and isoenzymes if total alkaline phosphatase is >ULN, and 5′nucleotidase if either GGT or total alkaline phosphatase is >ULN during study participation), total bilirubin, and albumin will be assessed.
Protein measurements using SOMAscan.
Markers of liver fibrosis: originally called Fibrotest®/Fibrosure®. The following will be assessed: α2-macroglobulin, apolipoprotein A1, total bilirubin, haptoglobin, GGT, and ALT.
NAFLD fibrosis score: The following formula will be utilized for the calculation of the NAFLD fibrosis score: −1.675+0.037×age (years)+0.094×BMI (kg/m2)+1.13×IFG (increased fasted glucose)/diabetes (yes=1, no=0)+0.99×AST/ALT ratio−0.013×platelet (×109/l)−0.66×albumin (g/dl).
Fasting insulin and glucose: Blood samples will be collected for fasting insulin and glucose assessment.
Liver biopsy: Subjects must have histologic evidence of NASH and liver fibrosis stage 2 or 3 (NASH clinical research network (CRN) staging criteria) demonstrated on liver biopsy within 6 months prior to randomization.

In addition, a Transient Elastography (FibroScan®) can be done at screening/baseline and at the Week 12, 24 and, 48.

Standard safety parameters and measures are collected including adverse events and serious adverse events according to definitions and process detailed in the protocol.

### Example 2: Safety, Tolerability and Efficacy of Licogliflozin, an SGLT1/2 Inhibitor in Patients with Non-Alcoholic Fatty Liver Disease: Interim Analysis of a Placebo-Controlled, Randomized Phase 2a Study

A randomized, double blinded, placebo-controlled Phase 2a study was conducted to evaluate the safety, tolerability and efficacy of licogliflozin in patients with either histologically confirmed NASH or with a biochemical phenotype suggestive of NASH.

Method: Patients with histologically confirmed NASH (F1-F3) or phenotypic NASH (BMI≥27 kg/m² in non-Asians or ≥23 kg/m² in Asians, ALT≥50 (males) or ≥35 (females) and type 2 diabetes (T2DM)) received daily oral licogliflozin at 150 mg, 30 mg or placebo in a 2:2:1 ratio for 12 weeks (NCT03205150). The primary endpoint is the effect on ALT level after 12 weeks of treatment. Secondary endpoints include improvement in body weight, liver fat content and AST, amongst others. The study size is 110 of which 77 have completed (placebo (n=18); licogliflozin 30 mg (n=25) and licogliflozin 150 mg (n=34)) and are included in the interim analysis.

Results: After 12 weeks of treatment, there was a 27% (17.2 U/L, p=0.036) and 19% (11.1 U/L, p=NS) placebo adjusted reduction from baseline levels of ALT at 150 mg and 30 mg, respectively. There was a reduction in AST of 30% (p=0.004) and 23% (p=0.043) as well as a 32% (p=0.001) and 26% (p=0.014) in GGT at 150 mg and 30 mg doses, respectively. Placebo adjusted reductions in body weight at both doses (˜4%, p=0.0001) and HbA1c (absolute change: 150 mg, 0.96% (p=0.0001); 30 mg, 0.81% (p=0.001)) were seen. Relative reduction in liver fat content was 22% (p=0.01) and 10% (p=NS) at 150 mg and 30 mg, respectively, and the proportion of patients with at least a 30% relative reduction was 66.7% (150 mg), 39.5% (30 mg) and 25% (placebo). Absolute reduction in liver fat was 4.45% (p=0.01) at 150 mg and 2.71% (p=NS) at 30 mg with 63.3% (150 mg), 43.5% (30 mg) and 18.8% (placebo) of patients achieving at least 5% absolute reduction. Diarrhea, the most common adverse event (AE), was reported by similar number of patients in the placebo and 30 mg group (38.9% vs. 40%) but was higher at the 150 mg dose (76.5%). Most diarrhea events (97.4%) were mild.

The study showed that Licogliflozin is safe and tolerable and improves multiple biochemical endpoints associated with NASH after 12 weeks of treatment. The study achieved its primary end-point of statistically significant reduction in ALT of at least 25% compared to placebo as showed above (mean relative decrease in ALT of 27% and 19% versus placebo at 150 mg and 30 mg, respectively and statistically significant reductions in AST and GGT versus placebo at both doses).

### Example 3: Dose-Dependent Reduction in Body Weight with Licogliflozin Treatment in Patients with Obesity Disease

This study was a randomized, double-blind, placebo controlled, dose finding study to evaluate the effect of licogliflozin (2.5, 10, 25 and 50 mg qd) in 126 Japanese patients with obesity disease. The primary objective was to examine the dose-response relationship of licogliflozin treatment in body weight reduction relative to placebo at 12 weeks. The secondary objectives included assessment of responder rates, change in parameters related to complications, visceral and subcutaneous fat area, and safety through 12 weeks of treatment.

RESULTS: The placebo-subtracted percentage change in body weight from baseline at Week 12 was −1.99, −3.00, −3.54, and −3.91% in licogliflozin 2.5, 10, 25 and 50 mg qd dose groups, respectively. In total, ≥50% of patients achieved reduction of ≥3% in body weight in licogliflozin 10, 25 and 50 mg qd dose groups versus placebo (7.1%; p≤0.002 for all). Treatment with licogliflozin was safe with no ketoacidosis and no new safety signals. Dual inhibition of SGLT1/2 with licogliflozin treatment induced a dose-dependent reduction in body weight in Japanese patients with obesity disease. Administration of licogliflozin (2.5, 10, 25 and 50 mg qd) over 12 weeks was safe and well tolerated in this study.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-16. (canceled)

17. A method for the treatment or prevention of a liver disease in a subject in need thereof, comprising administering once daily to said subject a therapeutically effective amount of a SGLT inhibitor, wherein the SGLT inhibitor is administered in the evening.

18. A method for the treatment, stabilization or lessening the severity or progression of a non-alcoholic fatty liver disease (NAFLD) or an intestinal disease in a subject in need thereof, comprising administering once daily to said subject a therapeutically effective amount of a SGLT inhibitor, wherein the SGLT inhibitor is administered in the evening.

19. The method of claim 18, wherein said non-alcoholic fatty liver disease (NAFLD) is non-alcoholic steatohepatitis (NASH).

20. A method for slowing, arresting, or reducing the development of a chronic liver disease or disorder in a subject in need thereof, comprising administering once daily to said subject a therapeutically effective amount of a SGLT inhibitor, wherein the SGLT inhibitor is administered in the evening.

21. A method for reducing cirrhosis or fibrosis in a subject having a disease that is non-alcoholic fatty liver disease (NAFLD) or non-alcoholic steatohepatitis (NASH), comprising administering once daily to said subject a therapeutically effective amount of a SGLT inhibitor, wherein the SGLT inhibitor is administered in the evening.

22. The method according to claim 17, wherein said method further comprises lack of worsening of the subject's NAFLD as defined by Activity (NAS) score, lack of worsening of the subject's Steatosis, Activity and Fibrosis (SAF) Activity score, reduction of liver fat in said subject, improvement in subject's Steatosis, improvement in subject's ballooning, NAFLD resolution, NAFLD resolution without worsening of fibrosis, reduction of fibrosis without NAFLD worsening, reduction of ALT levels in said subject, reduction of AST levels in said subject, reduction of HbA1c levels in said subject, lack of subject's progression to Cirrhosis, inhibiting progression of Non-Alcoholic Fatty Liver Disease (NAFLD) and/or Non-Alcoholic Steatohepatitis (NASH), or any combination thereof.

23. The method according to claim 17, wherein the SGLT inhibitor is an SGLT 1/2 inhibitor.

24. The method of claim 17, wherein the SGLT inhibitor is selected from licogliflozin, dapagliflozin, canagliflozin, empagliflozin, ipragliflozin, ertugliflozin, mizagliflozin, and sotagliflozin.

25. The method according to claim 24, wherein the SGLT inhibitor is licogliflozin.

26. The method according to claim 25, wherein licogliflozin is administered at a daily dose from about 30 mg to about 150 mg, preferably at about 30 mg.

27. The method according to claim 17, wherein diarrhea associated with administration of the SGLT inhibitor is reduced.

28. The method according to claim 17, wherein said method comprises resolution of steatohepatitis.

29. The method according to claim 17, wherein said method comprises improvement in liver fibrosis.

30. The method according to claim 17, wherein said method comprises resolution of steatohepatitis and improvement in liver fibrosis.
